(19) United States
(12) Patent Application Publication
(10) Pub. No.: US 2005/0090440 A1
(43) Pub. Date: Apr. 28, 2005

(54) Tumour treating combinations, compositions and methods

(76) Inventors: Geoffrey Wayne Krissansen, Auckland (NZ); Xuying Sun, Jinan (CN); Molly Frances Vale, Auckland (NZ)

Correspondence Address: FISH & RICHARDSON PC, 225 FRANKLIN ST, BOSTON, MA, 02110, US

(21) Appl. No.: 10/833,488

(22) Filed: Apr. 28, 2004

(30) Foreign Application Priority Data
Apr. 28, 2003 (NZ) 525560

(51) Int. Cl. A61K 38/17

(52) U.S. Cl. 514/12

(57) ABSTRACT

The present invention relates to the treatment of tumors, and the induction of anti-tumor immunity. The invention also relates to compositions and methods of use of same. In one aspect the invention combines the use of B7-1 and B7-H3 (or agents adapted in use to increase these molecules).

### FIELD

The present invention relates to the treatment of tumors. The invention also relates to compositions and methods of use of same in the treatment of tumours.

### BACKGROUND

The mechanisms involved in cellular activation, growth, proliferation and differentiation are complex involving the spatial and temporal interaction of many molecules. In light of this complexity there has been difficulty identifying methods of regulating cellular growth which may provide adequate therapies for the treatment or amelioration of aberrant cell growth, such as occurs in cancer.

Certain methods of regulating cell growth and proliferation as it relates to cancer have focused on the biological role of immune cells, particularly T cells, in targeting tumor cells for destruction.

For optimal activation, T cells must receive a costimulatory signal, in addition to T cell receptor (TCR) engagement. A plethora of different immunoglobulin (Ig)-like molecules are capable of delivering a costimulatory signal to stimulate T cell proliferation. One group of such molecules are the B7 family. The classical members of this family B7-1 (CD80) and B7-2 (CD86) interact with CD28 on T cells, and stimulate IL-2 production and the activation of naive T cells.¹ A number of new members of the B7 family have recently been identified, and the structures, expression, and functions of some elucidated.²⁻⁴

B7 signalling mechanisms are complex involving a number of different types of cells. While some B7 molecules up regulate an immune response, others may down regulate an immune response. Certain of the B7 molecules may be involved in primary immune responses, others in secondary responses. In addition, there appears to be diversity with respect to the type of cells within which different molecules within the B7 family are expressed.

B7 family members share ˜20% amino acid identity in their Ig variable (IgV) and Ig constant (IgC) extracellular regions. Whereas B7-1 and -2 are largely restricted to lymphoid tissue, the novel B7 family of ligands are much more broadly expressed in non-lymphoid tissue. They bind to receptors other than CD28, or CTLA-4, the alternative receptor for B7-1 and -2 which delivers an inhibitory signal. It has been proposed that they regulate the function and differentiation of effector lymphocytes in the periphery, but unlike B7-1 and -2, they do not prime naive T cells.⁴ B7-H2 (B7h, B7-related protein 1, GL50, LICOS) binds to inducible costimulator (ICOS) on T cells, and appears to play a major role in regulating Th2 responses.⁴⁻⁶ B7-H1 (PD-L1) and PD-L2 bind the receptor PD-1 on T cells, and inhibit T cell proliferation and cytokine production.^{7, 8} In support, PD-1-deficient animals suffer from autoimmune disorders, including lupus-like glomerulonephritis,¹⁰ and dilated cardiomyopathy.¹¹

The newest member of the B7 family, designated B7-H3, was cloned from a human dendritic cell-derived cDNA library.¹² It is widely expressed in various normal tissues, and its expression can be induced on monocytes and DCs. It appears to bind a counter-receptor on activated T cells that is distinct from CD28, CTLA-4, ICOS, and PD-1. B7-H3 is reported to costimulate the proliferation of CD4⁺ and CD8⁺ T cells, enhance the induction of cytotoxic T cells, and selectively enhance IFN-γ expression, with modest effects on TNF-α production.¹² B7-H3 is proposed to complement ICOS signaling by regulating Th1 and CTL responses. Essentially, little is known about the function of B7-H3, except that it enhances the induction of cytotoxic T cells, and selectively enhances IFN-γ expression.

Intratumoral gene transfer of mouse B7-1 and -2 has been shown to costimulate anti-tumour activity mediated by CD8⁺ T cells and NK cells, accompanied by augmented tumour-specific cytolytic T cell (CTL) activity involving both the perforin and Fas-ligand pathways.¹³⁻¹⁷

Chapoval et al¹² speculates that B7-H3 may also be a potential anti-cancer agent as it can induce many of the pathways required for a potent anti-tumour immune response. However, as Chapoval et al notes, B7-H3 is a poor costimulator of naive T cells, when compared to the costimulatory ability of B7-1. Accordingly, there has been some doubt as to whether B7-H3 could mount an adequate anti-tumour immune response, if at all. In addition, it has been argued that B7-H3 may play a critical role in the regulation of T cell responses after the initial priming stage.

There have been studies that have combined B7 molecules with cytokine treatment (Antonia et a²⁷), and integrin ligands (Cavallo et al²⁸), but to the inventor(s) knowledge, no one has tried combinations of B7 molecules in the treatment of cancer in vivo. While the latter studies indicated that combinations of different costimulators provided synergistic properties, other studies found that certain combinations impaired the anti-tumour immune response (Rafiee et al²⁹). This is a reflection of the complexity of T cell costimulation pathways.

To the inventors knowledge there has to date been no demonstration of the combined use of B7-H3 and B7-1 for the treatment of tumours in vivo. On the contrary the complexity of B7 signalling systems, little knowledge of the function of B7-H3, and the distinct characteristics and binding properties of B7-H3 compared to other B7 molecules may suggest that a therapy combining two or more such molecules may not be effective.

Bibliographic details of the publications referred to herein are collected at the end of the description.

Object

It is an object of the present invention to provide a therapy for the treatment of tumors or a method for inducing anti-tumor immunity and compositions suitable for use in such methods or at least to provide the public with a useful choice of either or both.

Statement of Invention

In accordance with the invention it has been surprisingly discovered and demonstrated that if B7-H3 is combined with the CAM B7-1 a significant reduction in the rate of growth of tumours and in many cases, complete eradication of tumours, results. The combined therapy is surprisingly applicable to established large tumours in addition to small tumours. The inventors believe the efficacy demonstrated is a result of an unexpected synergy between B7-H3 and B7-1.

The inventors have also demonstrated for the first time that B7-H3 alone, or in combination with B7-1, can induce anti-tumour immunity.

In one aspect, the present invention provides a method of treating tumors in a subject, the method comprising at least the steps of administering:
an effective amount of an agent adapted in use to increase B7-H3; and
an effective amount of an agent adapted in use to increase B7-1.

Preferably, the agent adapted to increase B7-H3 is B7-H3 or a functional equivalent thereof. More preferably, the agent adapted to increase B7-H3 is a nucleic acid vector adapted to express in use B7-H3 or a functional equivalent thereof.

Preferably, the agent adapted to increase B7-1 is B7-1 or a functional equivalent thereof. More preferably, the agent adapted to increase B7-1 is a nucleic acid vector adapted to express in use B7-1 or a functional equivalent thereof.

Preferably, the functional equivalent of B7-H3 is a polypeptide chosen from the group consisting: a polypeptide having at least approximately 40% amino acid sequence identity to the B7-H3 of GenBank accession number AF302102; or a polypeptide which is a fragment of the B7-H3 of GenBank accession number AF302102; and, wherein, the functional equivalent retains the ability to bind to natural ligands of B7-H3 involved in enhancing immune responses mediated by T cells.

Preferably, the functional equivalent of B7-1 is a polypeptide chosen from the group consisting: a polypeptide having at least approximately 40% amino acid sequence identity to the B7-1 of GenBank accession number NM_{—}005191 or P33681; or a polypeptide which is a fragment of the B7-1 of GenBank accession number NM_{—}005191 or P33681; and, wherein, the functional equivalent retains the ability to bind to natural ligands of B7-1 involved in enhancing immune responses mediated by T cells.

Preferably, the agents are administered intratumorally. Alternatively, the agents are administered systemically.

Preferably, the agents are administered sequentially in any order. Alternatively, the agents are administered simultaneously.

Preferably the subject is a mammal, more preferably the mammal is a human.

In another aspect, the invention provides a method of inducing anti-tumour immunity comprising at least the step of administering to a subject an effective amount of an agent adapted in use to increase B7-H3. Preferably, said agent is B7-H3, a functional equivalent thereof, or a nucleic acid vector adapted to express in use B7-H3 or a functional equivalent thereof.

In a further aspect, the present invention provides a method of treating tumors in a subject, the method comprising at least the steps of:
conducting a method as hereinbefore described:
isolating one or more immune cells from the subject;
expanding the one or more immune cells in vitro;
returning said immune cells to the subject.

Preferably, the one or more immune cells are splenocytes, lymph node lymphocytes, or tumour-infiltrating lympocytes.

Preferably, the immune cells are returned to the subject by injection.

In a further aspect, the invention provides a method of treating tumours comprising at least the steps of:
isolating one or more tumour cells from a tumour-bearing subject;
exposing one or more tumour cells to an effective amount of an agent adapted in use to increase B7-H3 and an agent adapted in use to increase B7-1; and
returning the one or more cells to the subject.

Preferably the one or more tumour cells are exposed to said agents simultaneously. Alternatively, the one or more tumour cells are exposed to said agents sequentially.

Preferably, the one or more isolated tumour cells are exposed to said agent by transfection with a nucleic acid vector adapted in use to express B7-H3 and B7-1, or functional equivalents thereof, or separate nucleic acid vectors adapted in use to express each separately.

Preferably, the one or more tumour cells are returned to the subject via injection.

In another aspect, the invention provides a composition comprising at least an agent adapted in use to increase B7-H3 and an agent adapted in use to increase B7-1 together with one or more pharmaceutically acceptable carriers, diluents or excipients.

In another aspect, the present invention provides the use of an agent adapted in use to increase B7-H3 and an agent adapted in use to increase B7-1 in the manufacture of a medicament for treating tumours.

Preferably the agent adapted to increase B7-H3 is B7-H3 or a functional equivalent thereof, more preferably the agent is a vector adapted to express B7-H3 or a functional equivalent thereof.

Preferably the agent adapted to increase B7-1 is B7-1 or a functional equivalent thereof, more preferably the agent is a vector adapted to express B7-1 or a functional equivalent thereof.

In a further aspect, the invention provides a kit comprising at least:
an agent adapted in use to increase B7-H3; and separately,
an agent adapted in use to increase B7-1.

In another aspect, the present invention provides a murine B7-H3 nucleic acid represented by SEQ ID NO:1, or a nucleic acid encoding the murine B7-H3 of SEQ ID NO:2.

In yet another aspect, the present invention provides a murine B7-H3 having the amino acid sequence of SEQ ID NO:2, or such sequence without the signal peptide.

The invention may also be said broadly to consist in the parts, elements and features referred to or indicated in the specification of the application, individually or collectively, in any or all combinations of two or more of said parts, elements or features, and where specific integers are mentioned herein which have known equivalents in the art to which the invention relates, such known equivalents are deemed to be incorporated herein as if individually set forth.

### FIGURES

These and other aspects of the present invention, which should be considered in all its novel aspects, will become apparent from the following description, which is given by way of example only, with reference to the accompanying figures, in which:

FIG. 1 Illustrates the results from the characterization of a mouse B7-H3 cDNA clone. (a) Nucleotide sequence of IMAGE clone #3483288 (SEQ ID NO:1), and deduced aa sequence (SEQ ID NO:2). The numbers in the right-hand margin refer to nucleotide and aa positions, respectively. The first nucleotide of the start codon and the initiator methionine have each been assigned position 1. The four potential asparagine (N) sites for N-linked glycosylation are emboldened. The signal peptide and transmembrane domains are underlined, whereas the IgV-like (light-line) and IgC-like (heavy-line) domains are overlined. The stop codon is represented by an asterisk. Conserved cysteine residues thought to form disulfide bonds of the IgV and IgC domains are emboldened. (b) Alignment of mouse B7H3 and B7-1 aa sequences. Several gaps (−) were introduced for optimal alignment. Identical aa are indicated by solid vertical lines, and aa with similar hydrophobicity are denoted by colons.

FIG. 2 Illustrates the results of the analysis of mouse B7H3 expression. (a) RT-PCR analysis of mouse B7H3 gene expression in multiple tissues. Primers annealing to sequences in the IgC-like and cytoplasmic domains generated a PCR product of 266 bp. Mouse G3PDH was PCR amplified as a positive control. (b) Engineered expression of Flag-B7-H3 in tumors. Tumors 0.4 cm in diameter were injected with empty vector (pcDNA3), or Flag-B7H3 expression vector (Flag-mB7-H3). Illustrated are representative tumor sections prepared 2 days following plasmid injection, stained brown with a mAb against the Flag tag (100× magnification). (c) Western blot analysis of expression of Flag-B7-H3 in tumors. Small (0.15 cm)(lane 2), and large (0.4 cm)(lane 3) tumors, injected 2 days earlier with a Flag-B7-H3 plasmid, were homogenized and the homogenates Western blotted with an anti-Flag mAb. The 45 kDa Flag-B7-H3 protein was present at similar levels in small and large tumors, where tubulin served as a marker to confirm that each lane contained similar amounts of tumor homogenate. Tumors injected with empty vector served as controls (lane 1).

FIG. 3 Illustrates that intratumoral injection of mouse B7-H3 plasmid eradicates small tumours. Established EL-4 tumours, approximately 0.1-0.25 cm in diameter, were injected at day 0 with 60 μg of expression plasmids encoding either mouse B7-H3 (a), Flag-tagged mB7-H3 (b), or mouse B7-1 (c), or an empty vector as control. Numbers in parentheses refer to the proportion of mice in a treatment group represented by the data set.

FIG. 4 Illustrates that intratumoral injection of mouse B7-H3 plasmid slows the growth of large tumours. Established EL-4 tumours, approximately 0.3-0.4 cm in diameter, were injected at day 0 with 100 μg expression plasmids encoding either mouse B7-H3, Flag-tagged mB7-H3, or mouse B7-1, or an empty vector control, as indicated. Each experiment group has 6 mice.

FIG. 5 Illustrates that mouse B7-H3-mediated anti-tumor immunity is largely mediated by CD8⁺ T cells and NK cells. Mice were treated with anti-CD4 (GK1.5), anti-CD8 (53-6.72), and the anti-NK cell (PK136) mAbs 4 days before intratumoral injection of B7-H3 plasmid, and every alternate day for the duration of the experiment. Rat IgG served as a control antibody. *Indicates a significant difference at P <0.05 from the rat IgG control group. Anti-CD8 and NK cell mAbs impaired anti-tumor immunity, which led to more rapid growth of tumors. Each experiment group has 6 mice.

FIG. 6 Illustrates that timed intratumoral gene transfer of B7-H3 and B7-1 plasmids induces stronger anti-tumour immunity than B7-H3 or B7-1 monotherapies. Established EL-4 tumors, approximately 0.35-0.45 cm in diameter, were injected at day 0 with 100 μg of expression plasmids encoding either mouse B7-H3, B7-1, or a combination of B7-H3 and B7-1. For combination therapy B7-H3 plasmid was injected first followed by B7-1 plasmid, however similar results were achieved when the order of injection was reversed (data not shown). Control tumours were injected with empty vector. Numbers in parentheses refer to the proportion of mice in a treatment group represented by the data set.

FIG. 7 Illustrates a comparison of the anti-tumour cytolytic activity generated by B7 immunotherapy. (a) Comparison of the anti-tumour CTL activity generated by gene transfer of either B7-H3, B7-1, or a combination of B7-H3 and B7-1 plasmids. Splenocytes obtained from mice 21 days following intratumoral injection of B7-1, B7-H3, or a combination of B7-H3 and B7-1 plasmids were tested for cytolytic activity against parental EL-4 tumor cells. The percentage cytotoxicity is plotted against various effector to target (E:T) ratios. Control animals received empty vector. (B) Adoptive transfer of anti-tumour CTL from treated mice eradicates small tumours. Splenocytes (2×10⁸) obtained as above from mice whose tumours had been injected with either B7-1 or B7-H3 plasmids, or empty vector control were adoptively transferred by intratumoral and i.p. injection into recipient mice bearing established tumours (˜0.1 cm in diameter). The sizes (cm) of tumours were monitored for 21 days following adoptive transfer. Complete tumor regression is denoted by vertical arrows. Mice were euthanased if tumors reached more than 1 cm in diameter (denoted by stars). (C) Splenocytes (2×10⁸) obtained as above from mice whose tumours had been injected with either B7-1, B7-H3, or a combination of B7-H3 and B7-1 plasmids, or empty vector control were adoptively transferred by intratumoral and i.p. injection into recipient mice bearing established tumours (0.35-0.45 cm in diameter). The sizes of tumours were monitored for 21 days following adoptive transfer, where the days are indicated as in FIG. 6b. Mice were euthanased if tumors reached more than 1 cm in diameter (denoted by stars). * Indicates a significant difference at P<0.05 from control groups of mice. ** Indicates a highly significant difference at P<0.01 between the combination therapy with B7-H3 and B7-1 plasmids, and the B7-1, or B7-H3 monotherapy.

FIG. 8 Illustrates that B7-H3 facilitates tumour cell lysis by anti-tumour CTL. Splenocytes from mice with B7-1 plasmid-treated tumours were mixed with disaggregated EL-4 cells that had been isolated 2 days after gene transfer from the tumours of mice injected with either B7-H3, or B7-1 plasmids, or a combination of B7-H3 and B7-1 plasmids. Cytotoxicity assays were performed, where * indicates a significant difference at P<0.01 from the empty vector injected control group, and ** indicates a significant difference at P<0.01 between the B7-H3/B7-1 combinational treatment and the respective monotherapies.

FIG. 9 Illustrates that antisense HIF-1α synergizes with B7-H3 to eradicate large tumours. Established tumors approximately 0.4 cm in diameter were injected at day 0 with either B7-H3 or antisense HIF-1α (aHIF-1) plasmids, a combination of B7-H3 and antisense HIF-1α plasmids; or empty vector. For the combination therapy the B7-H3 plasmid was injected first, followed 48 h later by the antisense HIF-1α plasmid. The sizes (cm) of tumours was recorded following gene transfer. Numbers in parentheses refer to the proportion of mice in a treatment group represented by the data set.

### DETAILED DESCRIPTION OF THE INVENTION

The following is a description of the preferred forms of the present invention given in general terms. The invention will be further elucidated from the Examples provided hereinafter.

Members of the B7 family costimulate the proliferation of lymphocytes during the initiation of antigen-specific humoral and cell-mediated immune responses. Whereas B7-1 and -2 are restricted to lymphoid tissues, and activate naive T cells, recently identified members including B7-H2 and -H3 are widely expressed on non-lymphoid tissues, and appear to regulate effector lymphocytes in the periphery.

B7-H3 has properties which may suggest it would display anti-tumour activity, including the ability to stimulate Th1 and cytotoxic T cell responses. However, B7-H3 is a poor costimulator of naive T cells, when compared to the costimulatory ability of B7-1. Further, it is widespread on non-lymphoid tissue, suggesting it is not involved in the initial priming stage but is possibly involved in the regulation of T cell responses post priming.

The inventors present studies on tumour growth in mice reveal that administration of B7-H3 is not as efficacious as expected or desired. The results identified that intratumoural injection of an expression plasmid encoding a newly described mouse homologue of B7-H3 was able to eradicate small (0.1 to 0.25 cm in diameter) EL-4 lymphomas in 50% of mice tested. When tested in large tumours (˜0.35 cm in diameter) B7-H3 failed to cause complete tumour regression, although it did appear to hold the growth of the tumours in check. In addition, as exemplified hereinafter, following B7-H3 plasmid treatment mice in which tumours completely regressed resisted a challenge with parental tumour cells, indicating systemic immunity had been generated.

The inventors studies indicate that B7-H3-mediated anti-tumour immunity is mediated by CD8⁺ and NK cells, with no apparent contribution from CD4⁺ T cells. The inventors investigated further to assess whether combining B7-H3 with other agents may provide for more effective tumour treatment options. As exemplified hereinafter, the inventors surprisingly found that B7-H3 synergizes with B7-1 to eradicate tumours, particularly large tumours. Timed gene transfer of a combination of B7-H3 and B7-1 induced strong anti-tumour immunity, resulting in the complete rejection of large tumours that were refractory to B7-H3 and B7-1 monotherapies. Anti-tumour immunity was systemic, and could be adoptively transferred.

The inventors believe these unexpected findings can be applied to the treatment of tumours or the inducement of anti-tumour immunity in mammals. Accordingly, in one embodiment the invention relates to a method of inducing anti-tumour immunity and/or treating tumours in a subject, the method comprising at least the step of administering an effective amount of an agent adapted in use to increase B7-H3 and an effective amount of an agent adapted in use to increase B7-1.

As used herein the terms “treating tumours” or “treatment” should be interpreted in their broadest possible context. The terms should not be taken to imply that a subject is treated until total recovery. Accordingly, “treatment” broadly includes amelioration of the symptoms or severity of a particular disorder, for example reduction in the rate of growth of a tumour, regression of a tumour, or preventing or otherwise reducing the risk of metastisis or of developing further tumours. The term should also be taken to encompass induction of anti-tumour immunity.

As used herein, a “therapeutically effective amount”, or an “effective amount” is an amount necessary to at least partly attain a desired response. A person of ordinary skill in the art will be able without undue experimentation, having regard to that skill and this disclosure, to determine an effective amount of a compound of this invention for a given disease or tumour.

A “subject” in accordance with the invention is an animal, preferably a mammal, more preferably a human.

A method of the present invention is applicable to any type of tumour, particularly weakly immunogenic tumours. It is also applicable to the treatment of both small and large or established tumours. In the context of the present invention, a small tumour may be considered, for example, as one that can generally be eradicated by immunotherapy with a single type of B7 molecule. A large tumour may be considered, for example, as one which is generally resistant to immunotherapy with a single type of B7.

“An agent adapted in use to increase” B7-H3 or B7-1 is an agent able to increase expression of, levels of, or activity of either molecule. In accordance with a preferred embodiment of the invention an “agent adapted in use to increase B7-H3” is B7-H3 itself a functional equivalent thereof, or a nucleic acid adapted in use to express B7-H3 or a functional equivalent thereof. Similarly, in a preferred embodiment of the invention an “agent adapted in use to increase B7-1” is B7-1 itself, a functional equivalent thereof, or a nucleic acid adapted in use to express B7-1 or a functional equivalent thereof.

Human B7-H3 has been described previously, for example see reference 12 hereinafter and US 20030119076 or WO 0118021. Exemplary human B7-H3 nucleic acid and amino acid sequences are published in GenBank under accession number AF302102. Exemplary murine B7-H3 nucleic acid and amino acid sequences are provided in GenBank under accession numbers Ay190318.

Human B7-1 has been described previously, for example see Greenfield et al³², Freeman et al³³, and WO 02/00717. An exemplary human B7-1 amino acid sequence is published in GenBank under accession number P33681. Exemplary human B7-1 nucleic acid and amino acid sequences are also found on GenBank under accession number NM_{—}005191. Exemplary murine B7-1 nucleic acid and amino acid sequences are provided in GenBank under accession number AF065896, or X60958. In addition the rat B7-1 amino acid sequence is found under accession number 507873.

It should be appreciated that reference to B7-H3 or B7-1, and their exemplary sequences provided on public databases (as mentioned above), should be taken to include reference to mature B7-H3 or B7-1 polypeptides excluding any signal or leader peptide sequences or other sequences not present in the mature protein that may be represented on such databases. Persons of general skill in the art to which the invention relates will readily appreciate such mature proteins.

As used herein, a “functional equivalent” of B7-H3 or B7-1 includes polypeptides and other molecules (which may be referred to herein as mimetics or analogues) capable of substantially displaying one or more known functional activities of the full-length or native B7-H3 or B7-1 proteins. In the context of the present invention functional equivalents will preferably retain an ability to bind to the natural ligands of B7-H3 or B7-1 which are involved in enhancing immune responses mediated by T cells, particularly the anti-tumor immunity demonstrated herein. Such function will preferably involve the increase in production and/or activity of anti-cancer cytotoxic T cells and/or enhance the natural killer cell-mediated killing of tumor cells. In the case of a functional equivalent of B7-1 it will preferably retain the ability to bind to CD28, and/or interact with NK cells. In the case of a functional equivalent of B7-H3 it will preferably retain the ability to bind to its immunostimulatory T cell receptor, and/or interact with NK cells.

It should be understood that “functional equivalents” of B7-H3 or B7-1 include polypeptides in which conservative amino acid substitutions have been made compared to the published amino acid sequence data for these molecules. Persons of general skill in the art to which the invention relates will appreciate appropriate conservative amino acid changes or substitutions having regard to established rules in this regard. The term “functional equivalents” is intended to include allelic variants and homologues of B7-H3 and B7-1. “Functional equivalents” should also be understood to include polypeptides in which one or more amino acids have been substituted in order to enhance function and/or expression.

In addition, “functional equivalents” should be taken to include those polypeptides having at least approximately 40% amino acid sequence identity to published full length B7-H3 or B7-1 amino acid sequences. More preferably, functional equivalents will have greater than or equal to approximately 60% amino acid sequence identity and even more preferably approximately greater than or equal to 70%. More preferably, the functional equivalents will have at least approximately 80%, 85%, 90%, 95% or 99% amino acid sequence identity to published B7-H3 or B7-1 amino acid sequences.

Fragments of the full length polypeptides of B7-H3 or B7-1 should also be taken to fall within the scope of “functional equivalents” of these molecules. Polypeptide fragments which retain the ligand-binding domains of the native protein may be of particular use in the present invention. Further examples of peptide fragments are described for example in US 20030119076 and WO 01/18021 (in the case of B7-H3) and in WO 02/00717 (in the case of B7-1); those representing fragments of the extracellular domain of B7-1 or B7-H3 may be of particular use.

B7-H3 and B7-1 and their functional equivalents of use in the invention include polypeptides which have been chemically modified. For example peptides may be modified by acetylation, glycosylation, cross-linking, disulfide bond formation, cyclization, branching, phosphorylation, conjugation or attachment to a desirable molecule (for example conjugation to bispecific antibodies), acylation, ADP-ribosylation, amidation, covalent attachment of a lipid or lipid derivative, covalent attachment of phosphotidylinositol, demethylation, formation of covalent cross-links, formation of cysteine, formation of pyroglutamate, formylation, gamma-carboxylation, GPI anchor formation, hydroxylation, methylation, myristoylation, oxidation, pegylation, proteolytic processing, prenylation, racemization, sulfation or otherwise to mimic natural post-translational modifications or to aid in presentation, for example. Functional equivalents also include peptides in which one or more amino acid of the natural protein is replaced with one or more non-naturally occurring amino acids. Proteins or peptides of use in the invention may be modified to allow for targeting to specific cells or cell membranes (for example, B7-H3 and/or B7-1, or functional equivalents thereof, may preferably be adapted to target the surface of tumour cells and insert or attach thereto). Fusion proteins are also included. Persons of general skill in the art to which the invention relates may appreciate other suitable modifications of use.

Mimetics or analogues of B7-H3, B7-1 or polypeptides thereof include for example peptiomimetics, a B7 mimetic phage isolated by phage library screening, and nucleic acid aptamers (see for example Burgstaller et al³⁴).

Functional equivalents of B7-1 or B7-H3 may be readily identified using standard methodology having regard to the description of the invention described herein. By way of example, the ability of a functional equivalent to bind to a natural ligand of B7-1 or B7-H3 may be tested using in vitro binding assays including for example ligand overlay, binding T cells in competition with recombinant B7-1 and/or B7-H3, or ELISA. Such techniques will be appreciated by persons of ordinary skill in the art to which the invention relates. However, exemplary techniques may be found in, Joseph Sambrook—Molecular Cloning: A Laboratory Manual; Antibodies: A Laboratory Manual by Ed Harlow (Editor), David Lane (Editor). In addition, in vitro stimulation assays in which the functional equivalent is tested in combination with an anti-CD3 mab to costimulate T cell proliferation may be used. Suitable stimulation assays are described in Chapoval A et al¹² and Lehnert et al³⁵ for example. Further, functionality may be tested in vivo using an animal model for example, as described herein after in the section entitled “Examples”.

Proteins and polypeptides (for example B7-H3, B7-1 or peptide functional equivalents) may be isolated and purified from natural sources, derived by chemical synthesis or genetic expression techniques (as are outlined broadly herein after), all of which are readily known in the art to which the invention relates. The inventor's also contemplate production of B7-H3 and B7-1 or peptide functional equivalents by an appropriate expression system, including transgenic animals.

In a preferred embodiment, proteins and polypeptides of use in the invention are produced via recombinant techniques. Suitable nucleic acid cloning and expression constructs will readily be appreciated by persons of general skill in the art to which the invention relates having regard to the published nucleic acid sequence data for the genes encoding B7-H3 and B7-1. Details of exemplary human genetic sequences are provided on GenBank as hereinbefore detailed. Suitable murine sequences may be as provided hereinafter or as published by Sun et al¹⁸ and on GenBank as described previously herein. Of course, having regard to the degeneracy in the genetic code, those skilled in the art will appreciate alternative sequences which may be of use in the invention; for example those sequences wherein certain nucleotides are substituted for alternative nucleotides without altering the amino acid sequence of the resultant product, or nucleotide substitutions which may result in conservative amino acid substitutions. The use of allelic variants and homologues of the above public nucleic acids sequences are also contemplated.

Nucleic acid constructs of use in producing proteins and polypeptides of use in the invention will generally contain heterologous nucleic acid sequences; that is nucleic acid sequences that are not naturally found adjacent to the nucleic acid sequences of the invention. The constructs or vectors may be either RNA or DNA, either prokaryotic or eukaryotic, and typically are viruses or a plasmid. Suitable constructs are preferably adapted to deliver a nucleic acid of the invention into a host cell and some may be capable of replicating in such cell. Recombinant constructs may be used, for example, in the cloning, sequencing, and expression of nucleic acid sequences relating to B7-H3 or B7-1.

Those of general skill in the art to which the invention relates will recognise many constructs suitable for use in cloning and expressing proteins and peptides of relevance to the invention. A recombinant construct or vector may be generated via recombinant techniques readily known to those of ordinary skill in the art to which the invention relates.

In the case of expression constructs, the inventors contemplate the use in the present invention of vectors containing regulatory sequences such as promoters, operators, repressors, enhancers, termination sequences, origins of replication, and other appropriate regulatory sequences as are known in the art. Further, the vectors may contain secretory sequences to enable expressed proteins or peptides to be secreted from a host cell. In addition, the expression vectors may contain fusion sequences which lead to the expression of inserted nucleic acid sequences of the invention as fusion proteins or peptides.

In accordance with the invention, transformation (or transfection) of a construct into a host cell can be accomplished by any method by which a nucleic acid sequence can be inserted into a cell. For example, techniques include transfection, electroporation, microinjection, lipofection, bolistic-bombardment, and adsorption.

As will be appreciated, transformed (or transfected) nucleic acid sequences of the invention may remain extrachromosomal or can integrate into one or more sites within a chromosome of a host cell in such a manner that their ability to be expressed is retained.

Any number of host cells known in the art may be utilised in cloning and expressing peptides and proteins of use in the invention. For example, plasmids may be cloned in E. coli strains, recombinant B7-H3 and B7-1 could be expressed in CHO (Chinese hamster ovary) cells using the pEE14 plasmid system, or in insect cells using baculoviral vectors.

Proteins and peptides of use in the invention may be recovered from a transformed (or transfected) host cell, or culture media, following expression thereof using a variety of techniques standard in the art. For example, detergent extraction, osmotic shock treatment and inclusion body purification. The protein may be further purified using techniques such as affinity chromatography, ion exchange chromatography, filtration, electrophoresis, hydrophobic interaction chromatography, gel filtration chromatography, and chromatofocusing.

As mentioned herein before, proteins and peptides of use in the invention may be in the form of fusion peptides or proteins; for example, fused with a peptide-based membrane translocating motif, fused with a motif which facilitates targeting to particular cell types, or alternatively, or in addition, fused with a motif which may aid in subsequent isolation and purification of the protein (for example, Ubiquitin, biotin, IgFc or histidine tags). Means for generating such fusion proteins are readily known in the art to which the invention relates, and include chemical synthesis and techniques in which fusion proteins are expressed in recombinant host cells, as may be above mentioned.

Proteins and peptides in accordance with the invention may also be conjugated to bispecific antibodies that may allow targeting to specific tumour cells. For example, the bispecific antibody may recognise a specific antigen on the surface of a target tumour, as well as an epitope on B7-H3 or B7-1. Persons of ordinary skill in the art to which the invention relates will recognise suitable techniques for achieving this end. However, by way of example, see Koumarianou et al³⁰.

In addition, B7-H3 and B7-1 and their functional equivalents may be conjugated to glycosylphosphatidylinositol (GPI) (or “pig-tails”) which would allow the protein to be inserted into the membrane of target cells in vivo, or in vitro, or to synthetic cell membranes in vitro. Techniques for achieving this are described for example in McHugh et ap³¹.

Techniques for chemical synthesis of proteins and peptides of relevance to the invention include “solid phase” chemical synthesis carried out by FMOC chemistry. Persons of general skill in the art may appreciate other appropriate techniques.

Techniques for chemically modifying polypeptides of the invention, or for generating mimetics or analogues will be appreciated by persons of general skill in the art to which the invention relates. However, exemplary techniques may be described in Creighton³⁶, Johnson³⁷, Seifter et al³⁸, or Rattan et al³⁹.

In a particularly preferred embodiment of the invention, the agent used to increase B7-1 or B7-H3 is a nucleic acid vector adapted in use to express these molecules or peptide functional equivalents. A suitable vector is exemplified hereinafter under the heading “Examples”. However, it will be appreciated that alternative nucleic acid vectors as may be known in the art, which will allow for delivery of a B7-H3 and/or B7-1 gene (or nucleic acid adapted to produce a functional equivalent of B7-H3 or B7-1), and subsequent expression of B7-H3 and/or B7-1 or their functional equivalent, can be used. For example, other naked plasmids that employ CMV promoters may be suitable. Viral vectors, such as adeno-associated virus (AAV) or lentiviruses for example may also be used. One advantage of using such viral vectors is that they may allow for systemic administration, as opposed to localised administration to a tumour.

Vectors adapted in use to express B7-H3 and/or B7-1 (or their functional equivalents) may be produced via standard recombinant techniques having regard to the published nucleic acid sequence data (and variant thereof) for such genes (as detailed herein before), of standard cloning and expression vectors, and of vectors adapted to deliver genetic material to a subject, or at least one target cell of a subject.

In the examples herein after, pCDNA3 was used. However, as mentioned herein before, the inventors contemplate the use of other naked plasmids that employ CMV promoters. In addition, viral vectors, such as adeno-associated virus (AAV) or lentiviruses may be used. As previously mentioned, the use of viral vectors supports systemic administration as opposed to localised administration to a tumour. Techniques standard in the art may be used to produce viral vectors of use in the invention. Briefly, these vectors are generated via standard recombinant techniques and packaged into viral particles for suitable administration to a subject (see for example, Ponnazhagan et al⁴⁰, Ponnazhagan et al⁴¹, Xu et al⁴²).

It should be appreciated that “vectors adapted in use to express or produce” in accordance with the invention may incorporate regulatory elements, such as promoters, enhancers, repressors and the like as known in the art, which may allow for the control and manipulation of the expression levels of proteins or peptides of relevance to the invention in use. For example, specific promoters, such as the early growth response gene-1 (Egr-1) promoter, may be used to maximise specific targeting and therapeutic efficacy. Egr-1 is transiently induced by a variety of extracellular stimuli such as hypoxia, or radiation.

The vectors may further contain sequences or elements which lead to the expression of B7-H3 and/or B7-1 (or functional equivalents thereof) as a fusion protein. For example, B7-H3 and/or B7-1 could be coupled to peptides which allow for targeting to specific tumour cells. In addition, it could be fused or coupled to an antibody directed to a specific tumour antigen.

In addition, the vectors may incorporate elements which allow for the permanent integration of genes encoding B7-H3 and/or B7-1 (or their functional equivalents) into the genome of at least one target cell of a subject.

It should be appreciated that vectors adapted in use to express B7-H3, B7-1, or their functional equivalents may include single vectors adapted to produce both, or separate vectors adapted to produce either B7-H3 or B7-1 or their functional equivalents. The vectors may also be adapted to express other proteins as may be desired.

The agents of the invention may be formulated, alone or in combination, into compositions with one or more pharmaceutically acceptable diluents, carriers and/or excipients. As-used herein, the phrase “pharmaceutically acceptable diluents, carriers and/or excipients” is intended to include substances that are useful in preparing a pharmaceutical composition, may be co-administered with an agent of the invention, while allowing same to perform its intended function, and are generally safe, non-toxic and neither biologically nor otherwise undesirable.

Those skilled in the art will readily appreciate a variety of pharmaceutically acceptable diluents, carriers and/or excipients which may be employed in preparing compositions in accordance with the invention. As will be appreciated, the choice of such diluents, carriers and/or excipients will be dictated to some extent by the nature of the agent to be used, the intended dosage form of the composition and the mode of administration thereof.

In the case of use of B7-H3 and B7-1 (or their functional equivalents) suitable liquid carriers, especially for injectable solutions, include water, aqueous saline solution, aqueous dextrose solution, and the like.

In addition, the inventors contemplate B7-H3, B7-1 and their functional equivalents being administered by a sustained-release system. Inasmuch as this is the case, compositions may include semi-permeable polymer matrices in the form of shaped articles, e.g., films, or microcapsules. Sustained-release matrices include polylactides, copolymers of L-glutamic acid and gamma-ethyl-L-glutamate, ethylene vinyl acetate, or poly-D-(−)-3-hydroxybutyric acid. Sustained-release compositions also include a liposomally entrapped compound. Compounds of this invention may also be PEGylated to increase their lifetime.

In the case of use of nucleic acids such as vectors adapted to express B7-H3, B7-1 and/or functional equivalents thereof in use, suitable carriers include water, aqueous saline solution, aqueous dextrose solution, and the like, with isotonic solutions being preferred for intravenous administration. As is mentioned elsewhere herein, the nucleic acid vectors of the invention may also be formulated into vehicles such as liposomes, which are especially suitable for administration of the nucleic acid vectors to tissues and tumours, or into biodegradable polymers such as poly (lactic acid), poly (lactide-co-glycolide) (PLGA), atelocollagen, or other polymers as non-viral gene delivery systems.

In the case of use of intratumoural injection of nucleic acids formulation into liposomes is preferred. Such formulation can be completed in accordance with the Examples herein after, or using alternative techniques standard in the art; by way of example, the techniques of Yang et al²⁶, and Kanwar et al¹³.

In a particularly preferred form of the invention, nucleic acid vectors are packaged into suitable viral particles, as mentioned hereinbefore.

In addition to standard diluents, carriers and/or excipients, a pharmaceutical composition comprising an agent of the invention may be formulated with additional constituents, or in such a manner, so as to enhance the activity of the agent or help protect the integrity of the agent. For example, the composition may further comprise constituents which provide protection against proteolytic degradation, or decrease antigenicity of the agent, upon administration to a subject. Alternatively, the agent may be modified so as to allow for tumour cell targeting as may be referred hereinbefore.

Additionally, it is contemplated that a composition in accordance with the invention may be formulated with additional ingredients which may be of benefit to a subject in particular instances.

As will be appreciated by those of ordinary skill in the art to which the invention relates, the agents of the invention and carriers, diluents or excipients may be converted to various customary dosage forms. In a preferred embodiment, the compositions are formulated into injectable liquids. However, alternative formulations such as orally administrable liquids, tablets, coated tablets, capsules, pills, granules, suppositories, trans-dermal patches, suspensions, emulsions, sustained release formulations, gels, aerosols, and powders may be used. Skilled persons will readily recognise appropriate formulation methods. However, by way of example, certain methods of formulating compositions may be found in references such as Gennaro AR: Remington: The Science and Practice of Pharmacy, 20th ed., Lippincott, Williams & Wilkins, 2000.

The inventors contemplate administration of any of the agents or compositions of the invention as abovementioned by any means capable of delivering the desired activity to a target site within the body of a subject. A “target site” is preferably the site of a tumour.

For example, administration may include parenteral administration routes, systemic administration routes, oral and topical administration. As will be appreciated, the administration route chosen may be dependent on the site of a tumour within a subject, as well as the nature of the agent or composition being used. However, in a preferred embodiment of the invention the agents or compositions are administered intratumourally via injection, optionally using ballistics. In another preferred form, the agents or compositions are administered systemically (for example orally, or via intravenous injection).

As will be appreciated, the dose of an agent or composition administered, the period of administration, and the general administration regime may differ between subjects depending on such variables as the severity of symptoms of a subject, the size of the tumour to be treated, the site of the tumour to be treated, the type of disorder or tumour to be treated, the mode of administration chosen, and the age, sex and/or general health of a subject. However, by way of general example, the inventors contemplate from approximately 60 micrograms to 60 milligrams per dose being appropriate for administration of nucleic acid vectors adapted in use to express B7-H3 and/or B7-1 by localised, or parenteral, injection. The dose may be repeated as desired.

It should be appreciated that administration may include a single daily dose or administration of a number of discrete divided doses as may be appropriate.

The agents or compositions abovementioned may be administered in accordance with a method of the invention sequentially, in any order, or simultaneously. Simultaneous administration includes administration of the agents in distinct formulations or compositions, or the agents together in a single formulation or composition. For example, in the case of use of nucleic acids adapted in use to express B7-H3, B7-1 or their functional equivalents, a single nucleic acid vector adapted to produce both may be utilised, separate vectors in a single formulation, or separate vectors in distinct formulations. In one preferred form of the invention, the agents or compositions are administered sequentially, 48 hours apart.

The inventors also contemplate the administration regimes which combine different modes or routes of administration. For example, intratumoural injection and systemic administration could be combined.

It should be appreciated that a method of the invention as above mentioned may further comprise additional steps such as the administration of additional agents or compositions which may be beneficial to a subject. A further example includes the excision of a tumour from a subject followed by administration of compositions or agents of the invention directly to the tissues that surrounded the tumour site. This technique may help prevent the growth of any tumour cells inadvertently left behind following tumour excision.

In another embodiment the invention provides a method of inducing anti-tumour immunity comprising at least the step of administering to a subject an effective amount of an agent adapted in use to increase B7-H3. Preferably, said agent is B7-H3, a functional equivalent thereof, or a nucleic acid vector adapted to express B7-H3 or a functional equivalent in use. Such agents may be made, formulated and administered as hereinbefore described.

In another embodiment, the invention provides a method of treating tumours in a subject which comprises at least the steps of: conducting any of the methods described above; isolating one or more immune cells from the subject; expanding the one or more immune cells in vitro; and, returning said immune cells to the subject.

Preferably, the one or more immune cells are splenocytes, lymph node lymphocytes, or tumour-infiltrating lymphocytes. These cells may be isolated from a subject according to standard techniques known in the art. For example, cells may be isolated following surgery.

Once harvested from the subject, the immune cells may be cultured and expanded using standard techniques and media. For example, see Keith et al⁴³. It is preferable that the tumour specificity of the cells is maintained during this process, which can be accomplished by stimulating cells with tumour fragments, antigen, or tumour peptides. The ex vivo expansion of immune cells in this manner may help complement and extend the subjects own population of cells during a period within which they may be immuno-compromised due to the presence of one or more tumours within their body.

The expanded immune cells may be returned to the subject by any means available for doing so. Most preferably, the cells are returned via injection.

In another embodiment the invention relates to method of treating tumours comprising at least the steps of: isolating one or more tumour cells from a tumour-bearing subject; exposing one or more tumour cells with an effective amount of an agent adapted in use to increase B7-H3 and an agent adapted in use to increase B7-1; and, returning the one or more cells to the subject.

The ex vivo method of this embodiment of the invention may be performed in accordance with standard procedures. Briefly, cells are harvested from a subject, the cells are cultured, exposed to agents in accordance with the invention, and maintained under conditions conducive to cellular viability and which allow the agent to act in its desired manner. Ordinarily skilled persons will appreciate appropriate cell culture conditions. However, by way of example, the techniques referred to in Singh et al⁴⁴, and Antonia et al⁴⁵ are of use to this end. The cells are preferably isolated from a subject by surgical excision of tumour material, and preparation of single cell suspension following enzyme digestion, such as with collagenase.

As used herein the term “exposing the one or more cells” should be taken in its broadest possible context. It is intended to include any means of delivering the agents to the cells. As will be appreciated, the means of exposure may vary depending on the nature of the agent concerned. For example, in the case of use of nucleic acids such as vectors adapted to express B7-H3, B7-1 or their functional equivalents standard transformation techniques may be used. For example, techniques involving liposomes, polymeric microparticles, lipofection, biolistic delivery, electroporation, viral infection, and calcium phosphate may be utilised. It will be appreciated that these techniques may result in permanent or transient expression of B7-H3, B7-1 or their functional equivalents.

The cells are returned to the subject by any known method. Preferably, the cells are returned via implantation or systemically, preferably by injection.

The methodology described in U.S. Pat. No. 6,183,734²⁴, or in Antonia et al²⁷, for example, may be utilised in effecting the above ex vivo-type methods of the invention.

In a further embodiment, the invention relates to a kit for the treatment of tumours and/or
for inducing tumour immunity in a subject, the kit comprising at least:
an agent adapted in use to increase B7-H3; and, separately,
an agent adapted in use to increase B7-1.

In another embodiment, the invention relates to a murine B7-H3 nucleic acid represented by SEQ ID:1, or a nucleic acid encoding the murine B7-H3 of SEQ ID NO:2. A further embodiment of the invention is a murine B7-H3 having the amino acid sequence of SEQ ID NO:2, or a B7-H3 having said amino acid sequence without the signal sequence.

### EXAMPLES

Materials and Methods

Characterization of Mouse B7-H3 cDNA, and Vector Preparation

IMAGE clone #3483288 (GenBank accession no. BE311080) was purchased from Invitrogen New Zealand Ltd, Penrose, Auckland, New Zealand. The plasmid was completely sequenced using facilities provided by the DNA Sequencing and Genotyping Unit, School of Biological Sciences, University of Auckland, Auckland. A 951 bp cDNA fragment encoding full-length mouse B7-H3 was released and subcloned into pcDNA3.1 (Invitrogen). DNA sequence encoding the Flag tag (DYKDDDDK) was fused to the N-terminal sequence of mouse B7-H3 via PCR, using B7-H3 cDNA as a template and the two primers (5′-GGAATTCAAGATGGTTACAAGGATGATGATGA TAAACTTCGAGGATGGGGTGGCCCCAGTG-3′ and 5′-GGGTGGGCCCCCCACCT GGGAAGG-3′). The Flag-B7-H3 cDNA was cloned via pGEMT (Promega Corporation) into pcDNA3.1. The integrity of all the constructs was confirmed by DNA sequencing. The expression plasmid B7-1-pCDM8, which contains a 1.2 kb cDNA fragment encoding full-length mouse B7-1 was constructed from a cDNA clone kindly provided by Dr P Linsley, Bristol-Myers-Squibb, Seattle, Wash., USA. [Kanwar, J. R., Berg, R. W., Lehnert, K., and Krissansen G. W. Taking lessons from dendritic cells: Multiple xenogeneic ligands for leukocyte integrins have the potential to stimulate anti-tumor immunity. Gene Therapy, 6: 1835-1844, 1999; Chen, L., Ashe, S., Brady, W. A., Hellstrom, I., Hellstrom, K. E., Ledbetter, J. A., McGowan, P., and Linsley, P. S. Costimlation of anti-tumour immunity by the B7 counterreceptor for the T lymphocyte molecules CD28 and CTLA-4. Cell 71: 1093-1102, 1992.]

RNA Analysis by RT-PCR

Total RNA was extracted with Trizol Reagent (Life Technologies, Inc. [GIBCO BRL], Rockville, Md.) from multiple mouse tissues, and a parental EL-4 tumor established in a C57BL/6 mouse. The RNAs were treated with RNase-free DNase I (Boehringer Mannheim, Mannheim, Germany), and reverse transcribed using Superscript II RNase H reverse transcriptase (Life Technologies) at 42° C. for 50 min. B7-H3 cDNA was PCR amplified with the primer pair 5′-CTCAGCTGCCTGGTACGCAA-3′ (nt 651-671 within the IgC like domain) and 5′-CAGAGGGTTTCAGAGGCCGTA-3′ (nt 916-896 within the cytoplasmic domain) for 30 cycles of 94° C for 30 s, 58° C for 30 s, and 72° C. for 30 s Mouse glyceraldehyde 3-phosphate dehydrogenase (G3PDH) used an internal control was PCR amplified with the primers G3PDHA (5′-TGAAGGTCGGTGTGAACGGA-3′) and G3PDHB (5′-CATGTAGGCCATGAGGTCCACCAC-3′), generating a 980 bp PCR product. PCR products were electrophoresed on a 1.5% agarose gel containing ethidium bromide, and visualized with UV light.

Mice and Cell Line

Female C57BL/6 mice, 6-8 weeks old, were obtained from the Animal Resource Unit, Faculty of Medicine and Health Science, University of Auckland, Auckland, New Zealand. The EL-4 thymic lymphoma, which is of C57BL/6 (H-2b) origin, was purchased from the American Type Culture Collection (Rockville, Md., USA). It was cultured at 37° C. in DMEM medium (GIBCO BRL, Grand Island, N.Y., USA), supplemented with 10% foetal calf serum, 50 U/ml penicillin/streptomycin, 2 mM L-glutamine, and 1 mM pyruvate.

Intratumoral Injection of Expression Plasmids and Measurement of Anti-tumour Activity

Plasmids were purified with cesium chloride and diluted in a solution of 5% glucose in 0.01% Triton X-100, and mixed in a ratio of 1:3 (wt:wt) with DOTAP cationic liposomes (Boehringer Mannheim, Mannheim, Germany), as described previously (13). The final plasmid concentration was 0.6 mg/ml for the treatment of small tumors and 1 mg/ml for larger tumors. Tumors were established by subcutaneous injection of 2×10⁵ EL-4 tumor cells into a site in the right flank of mice from which a small patch of fur had been removed. The growth of tumors was determined by measuring two perpendicular diameters. Animals were killed when tumors reached more than 1 cm in diameter, in accord with Animal Ethics Approval (University of Auckland). Tumors that had reached the expected size after approximately 14-18 days were injected with 100 μl expression plasmid solution at multiple sites. Empty vectors served as control reagents. Mice whose tumors completely regressed were rechallenged 3 weeks after the disappearance of tumors by injecting 1×10⁶ parental EL-4 cells subcutaneously into the opposing flank (left flank). All experiments included 6 mice per treatment group, unless specifically mentioned, and each experiment was repeated at least once. For combinational treatment, B7-H3, B7-1, and antisense HIF-l: plasmids were injected in a timed fashion, such that the second plasmid was injected 48 h after the first. Cured mice were rechallenged 3 weeks after tumor disappearance by injecting 2×10⁵ or 2×10⁷ EL-4 cells subcutaneously into the opposing flank (left flank).

Depletion of Leukocyte Subsets

Mice were depleted of CD8⁺, and CD4⁺ T cells and NK cells by i.p. and i.v. injection two days prior to intratumoral injection of expression plasmids, and thereafter every alternate day with 300 μg (0.1 ml) of the 53-6.72 (anti-CD8), Gk1.5 (anti-CD4), and PK136 (anti-NK) mAbs. Rat IgG (Sigma, St Louis, Mo.) was used as a control antibody. Antibodies were an ammonium sulphate fraction of ascites, which titered to at least 1:2000 by FACS (Becton Dickinson, San Jose, Calif., USA) staining of splenocytes. Depletion of individual leukocyte subsets was found to be more than 90% effective, as determined by FACScan analysis. Each experiment group has 6 mice.

Adoptive Transfer of Stimulated CTLs

Splenocytes isolated from donor mice that had been cured by treatment with either B7-H3 or B7-1 plasmids, or B7H3 plasmid in combination with B7-1 plasmid, were resuspended in Hank's balanced salt solution containing 1% FCS, and stimulated with 5 μg/ml PHA and 100 U/ml recombinant mouse IL-2 for 4-5 days. Recipient mice, bearing established tumours, received both intratumoral and i.p. injections of 2×10⁸ cultured splenocytes.

Cytotoxicity Assay

Splenocytes were harvested from mice 7 days after tumours had disappeared following intratumoral injection of either B7-1 or B7-H3 plasmid, or a combination of B7-H3 and B7-1 plasmids. Splenocytes (10⁶, 5×10⁵, 10⁵) were incubated at 37° C. with 1×10⁴ EL-4 target cells in graded E:T ratios in 96-well round-bottom plates. After a 4 h incubation, 50 μl of supernatant was collected, and lysis was measured using the Cyto Tox 96 Assay kit (Promega, Madison, Wis., USA). Background controls for non-specific target and effector cell lysis were included. After background subtraction, percentage of cell lysis was calculated using the formula: 100×(experimental-spontaneous effector-spontaneous target/maximum target-spontaneous target).

In vitro Killing Assay to Determine Whether B7-H3 Facilitates Tumour Cell Lysis

Tumours were excised 2 days following injection of tumours with either B7-H3 or B7-1 plasmids, or a combination of B7-H3 and B7-1 plasmids, and injected with collagenase. Tumour cells were isolated by homogenization, further collagenase treatment, and centrifugation. Splenocytes obtained from mice whose tumours had been injected with B7-1 plasmid were mixed with the disaggregated EL-4 cells at different effector of target ratios, as above, and subjected to a cytotoxicity assay as described above.

Immunohistochemistry

Tumour cryosections (10 μm) prepared 2 days after intratumoral injection of plasmids were treated with acetone, rinsed with PBS, blocked with 2% BSA for 2 h, and incubated overnight with a rabbit anti-Flag mAb (Sigma). They were subsequently incubated for 30 min with appropriate secondary antibodies, using the VECTASTAIN Universal Quick kit (Vector Laboratories, Burlingame, Calif., USA); and developed with Sigma FAST DAB (3,3′-diaminobenzidine tetrahydrochloride) and CoCl₂ enhancer tablets (Sigma). Sections were counterstained with Mayer's hematoxylin, mounted, and examined by microscopy.

Western Blotting

Tumours injected with expression plasmids were excised 2 days later, and homogenized in protein lysate buffer (50 mM Tris pH 7.4, 100 μM EDTA, 0.25 M sucrose, 1% SDS, 1% NP40, 1 μg/ml leupeptin, 1 μg/ml pepstatin A, and 100 μM PMSF). Homogenates were resolved by 10% SDS-PAGE, and proteins electrophoretically transferred to nitrocellulose membrane (Hybond C extra; Amersham Life Science England). Membranes were blocked with 3% BSA in TTBS (20 mM Tris, 137 mM NaCl pH 7.6 containing 0.1% Tween-20), and incubated with anti-Flag mAb (Sigma). They were incubated with horseradish peroxidase-conjugated secondary antibodies, and immunoreactivity was detected by Enhanced Chemiluminescence (Amersham International plc. England), and exposure to X-Ray film.

Statistical Analysis

Results were expressed as mean values±standard deviation (s.d.), and a Student's t test was used for evaluating statistical significance. A value less than 0.05 (P<0.05) was used for statistical significance.

Results

Cloning and Characterization of Mouse B7-H3

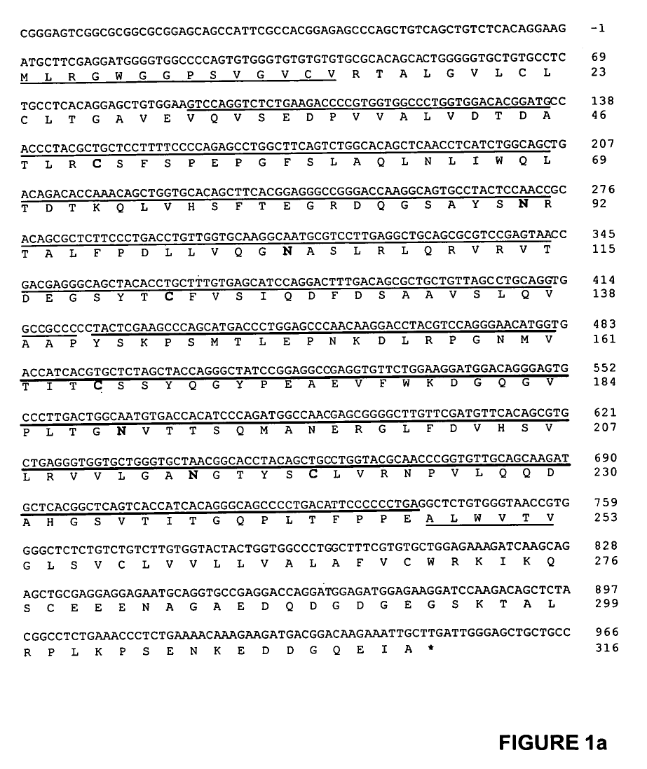

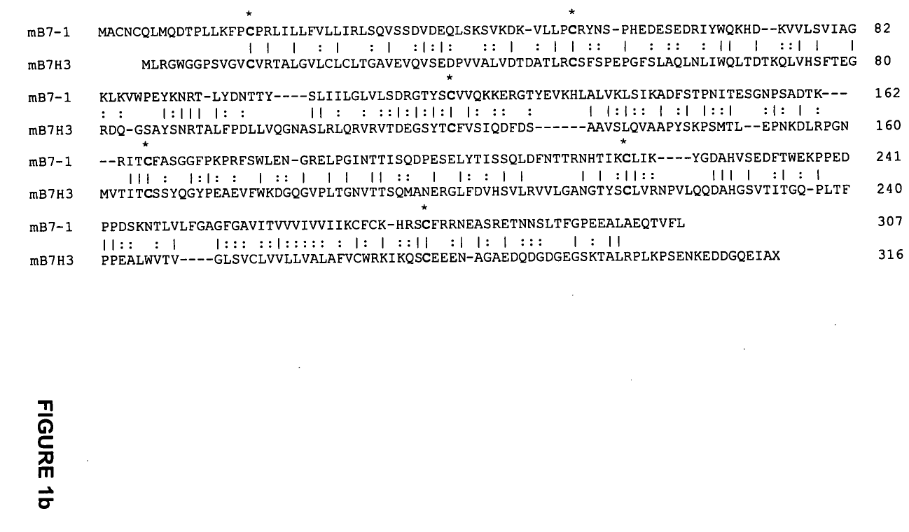

The mouse EST database of the National Center for Biotechnology Information (NCBI) was searched with cDNA sequences encoding the extracellular regions of B7-1 and -2, and identified an IMAGE clone (#3483288; Genbank accession no. BE311080), which encoded a full-length B7-like cDNA sequence with extensive similarity to the subsequently published sequence of human B7-H3.¹² The cDNA sequence of clone #3483288 encoded a 316 amino acid residue (aa) type I membrane protein, consisting of a 29 aa signal peptide, single 112 aa IgV-like and 106 aa IgC-like extracellular domains, a 24 aa transmembrane region, and a short 45 aa cytoplasmic tail (FIG. 1a). The encoded protein contains four potential N-glycosylation sites at aa positions 91, 104, 189, and 215 of the immature sequence. While this manuscript was in preparation, Sun et al.¹⁸ reported a near identical aa sequence obtained from a different EST clone (Genbank accession no. BF450618; IMAGE clone 3674228), and designated the encoded sequence as mouse B7-H3. There is one conservative aa difference within a short segment of the cytoplasmic tail between the sequence reported here (SCEEENAGAE), and that reported by Sun et al. (SCEEENSGAE), which may arise from a polymorphism. The cDNA sequence for mouse B7-H3 was not previously reported, or submitted in GenBank, and hence has been included here for completeness. Mouse B7-H3 shares 88% similarity with human B7-H3 (GenBank accession no. XM_{—}016883), compared to 26% similarity with mouse B7-1 (GenBank accession no. MMU278965) (FIG. 1b).

Expression of Endogenous Mouse B7-H3

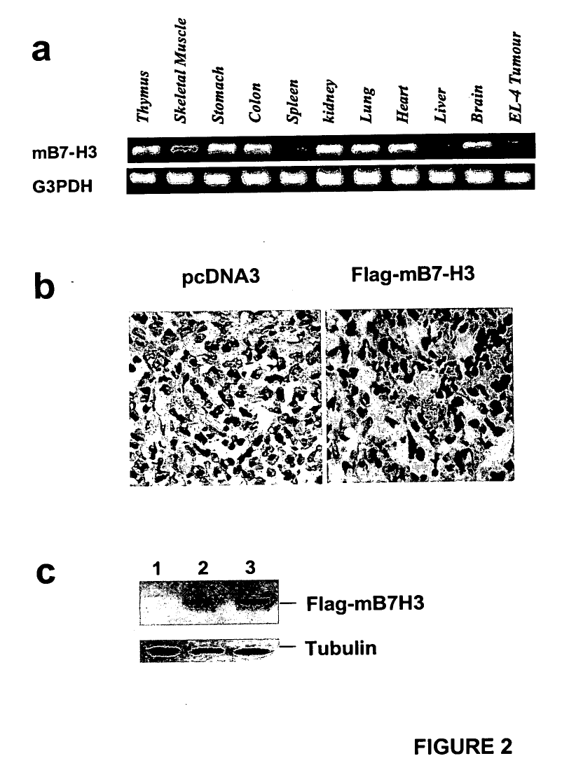

The expression of mouse B7-H3 mRNA in multiple tissues was examined by reverse transcription-polymerase chain reaction (RT-PCR), using a pair of primers that anneal to sequences encoding the IgC-like and cytoplasmic domains that are located on separate exons. Transcripts encoding mouse B7-H3 were widely expressed in all tissues examined (FIG. 2a). B7-H3 was not detectable in EL-4 tumor cells cultured in vitro (data not shown). RT-PCR showed that B7-H3 could be detected in solid EL-4 tumors, but at an extremely low level compared to expression in other normal tissues (FIG. 2a). The lowly expressed B7H3 detected in EL-4 tumors is presumably derived from normal vascular endothelial cells, or blood cells.

Intratumoral Gene Transfer Results in Expression of Mouse B7-H3 In Situ

Tests were conducted to establish whether or not mouse B7-H3 would induce anti-tumor immunity. A Flag tag was fused to the N-terminus of mouse B7-H3 in order to detect expression of mouse B7-H3 plasmids injected directly into tumors in situ. Tumors injected with 60 μg of Flag-B7H3/pcDNA3.1 expression plasmid were sectioned 2 days following gene transfer. The representative photographs (FIG. 2b) reveal exogenous expression of Flag-tagged B7-H3 throughout tumors, whereas control sections from vector-only treated tumors were not stained with the anti-Flag antibody (FIG. 2b). To determine whether the Flag-tagged B7-H3 transgene was as efficiently taken up and expressed by large versus small tumors, small (0.15 cm) and large tumors (0.4 cm) were injected with either 60 or 100 μg of Flag-B7H3/pcDNA3.1 expression plasmid, respectively, followed by homogenization 2 days later. Western blot analysis of tumor homogenates revealed that exogenous Flag-tagged B7-H3 was expressed in situ at similar levels in both small and large tumors (FIGS. 2c). As expected, control homogenates (lane 1) from vector-only-treated tumors were not stained with the anti-Flag tag antibody.

Gene Transfer of Mouse B7-H3 Eradicates Small EL-4 Lymphomas

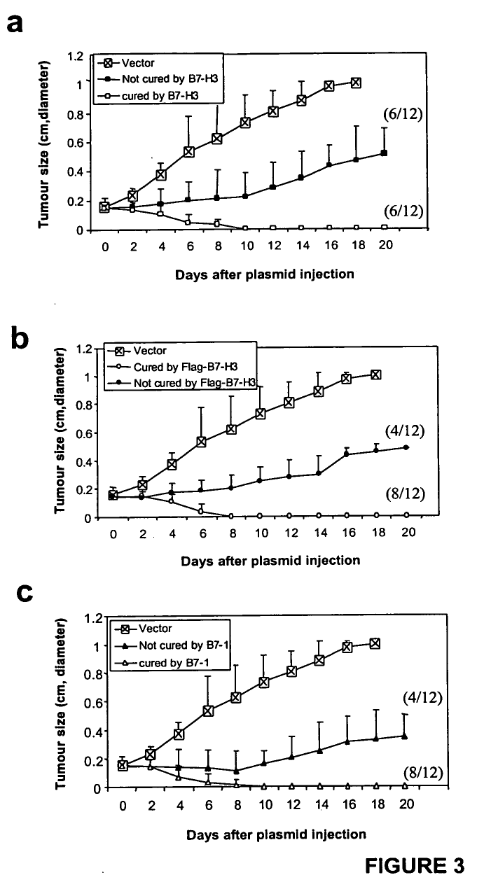

Small EL-4 tumors of 0.1-0.25 cm in diameter were established in C57BL/6 mice, and injected with a DNA/liposome transfection vehicle containing 60 μg of mouse B7-H3/pcDNA3.1 plasmid DNA (non-Flag-tagged version). Of a range (30-120 μg) of dosages tested, 60 μg proved to be the most effective against small tumors (data not shown), as found previously with a panel of plasmids encoding other T cell costimulators.¹³ Tumor growth was monitored for 20 days, and compared to the growth of tumors treated with 60 μg of empty vector control, or 60 μg of mouse B7-1 expression plasmid. Tumors grew rapidly in the control group, reaching 1 cm in size within 14-17 days of injection of empty plasmid, whereas tumors treated with the mouse B7-H3 plasmid rapidly and completely regressed in 50% (6/12) of mice. Tumours that did not regress completely were nevertheless significantly (P<0.01) slowed in their growth compared to tumors treated with empty plasmid (FIG. 3a, Table 1). Similar results were achieved with Flag-tagged mouse B7-H3 plasmids. Tumours completely regressed in 67% (8/12) of mice (FIG. 3b, Table 1). By comparison, tumours injected with mouse B7-1 completely regressed in 8 of 12 of mice, or were otherwise significantly (P<0.01) slowed in their growth (FIG. 3c, Table 1). Mice whose tumors had completely regressed were rechallenged with 1×10⁶ parental EL-4 cells, and remained tumor-free for a further 21 days (Table 1), indicating that systemic anti-tumour immunity activity had been established.

Large Tumours Suppress B7-H3-Mediated Anti-Tumour Immunity

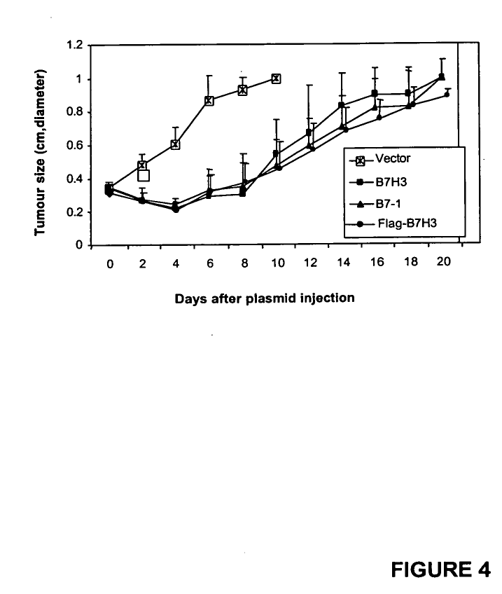

To determine whether B7-H3 therapy was equally effective against larger tumors, tumors approximately 0.35 cm in diameter were injected with 100 μg of either mB7H3/pcDNA3.1, Flag-tagged mB7-H3/pcDNA3.1, B7-1/pcDNA3.1, or empty vector. Although either B7H3 or its Flag-tagged version failed to cause complete tumor regression, tumor growth was nevertheless held in check for eight days, whereas control mice had to be euthanased (FIG. 4). Thereafter, tumors began to regrow reaching 1 cm in another 10 days. Thus, while not completely effective B7-H3 therapy could significantly (P<0.05) inhibit the growth of large tumors. The efficacy of B7-H3 plasmid therapy was similar to that achieved with B7-1 plasmids. The disparity in the effectiveness against small versus large tumors is not due to an inadequate dosage of mB7H3 expression vector, as there was little or no difference in the expression levels of the Flag-B7-H3 transgene in large versus small tumours (FIG. 2c), and increased doses of mB7H3 plasmid (up to 200 μg) were no more effective against large tumors than a dose of 100 μg (data not shown).

Anti-Tumour Immunity Induced by Mouse B7-H3 Largely Depends on CD8+ T Cells and NK Cells

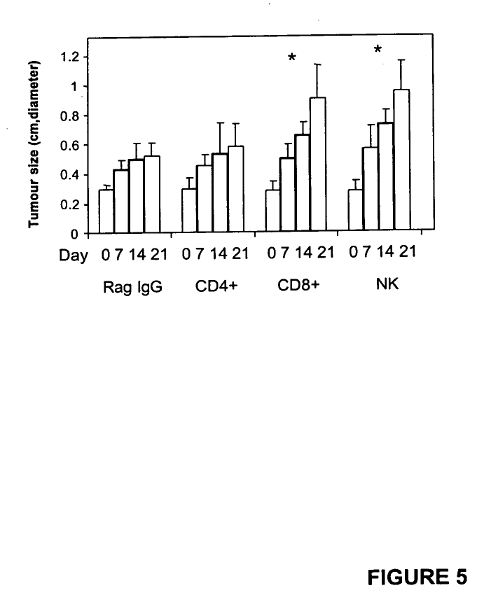

Leukocyte depletion analysis was carried out to identify immune cell subsets involved in mediating the anti-tumor immunity generated by B7-H3. Depletion of either CD8⁺ T cells or NK cells impaired anti-tumor immunity, leading on average to significantly (P<0.05) increased tumor growth, whereas depletion of CD4⁺ T cells had no affect (FIG. 5). Thus, B7-H3-mediated anti-tumor immunity against EL-4 tumors is largely dependent on CD8⁺ T cells and NK cells.

Timed Gene Transfer of B7-H3 and B7-1 Plasmids is Superior to Monotherapies

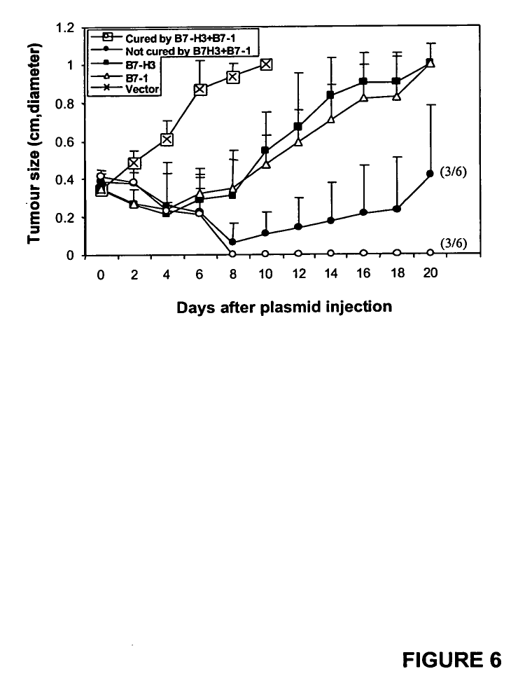

B7-H3 and B7-1 appear to activate different T cell subtypes. It was sought to determine whether they might synergize to induce heightened anti-tumour immunity. Tumours of 0.35-0.45 cm in diameter were injected with 100 μg each of B7-1, B7-H3, and empty expression plasmids, or a combination of B7-H3 and B7-1 plasmids where B7-H3 plasmid was injected first followed 48 h later by B7-1 plasmid, or vice versa B7-1 was injected followed by B7-H3 (only data for the former is shown). Tumours treated with B7-1 and mB7-H3 monotherapies were significantly (P<0.05) retarded in their growth compared to tumours injected with empty plasmid (FIG. 6). Tumours were held in check for 8 days before assuming growth rates identical to controls. No tumour was completely rejected. The inability to eradicate large tumours by B7-1 and B7-H3 monotherapies is not due to gene dosage, as increasing the dosage of plasmids to 200 μg had no greater affect. In complete contrast to the monotherpies above mentioned, combined immunogene therapy with B7-H3 and B7-1 expression plasmids was surprisingly much more successful, such that tumours were completely rejected in 50% of the mice (FIG. 6). Further, tumours that were not rejected grew more slowly (P<0.01) compared to tumours treated with B7-1 or B7-H3 monotherapies.

Combinational Treatment with B7-H3 and B7-1 Stimulates Stronger Anti-Tumour-Specific CTL Activity, which can be Adoptively Transferred to Cure Recipient Animals

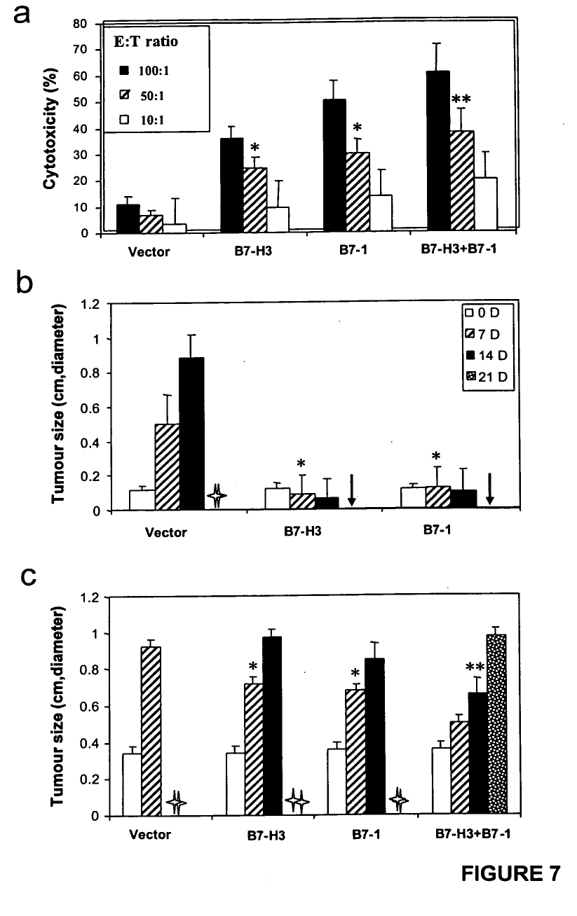

The anti-tumour CTL activity of splenocytes obtained 21 days following gene transfer was significantly (P<0.01) augmented in mice whose tumours had been injected with B7-H3 and B7-1 expression plasmids, versus those that received empty vector (FIG. 7A). The anti-tumour CTL activity of splenocytes was highest in mice whose tumours were injected with a combination of B7-H3 and B7-1 expression plasmids. All the mice treated with B7-H3, B7-1, or a combination of B7-H3 and B7-1 resisted a challenge with 2×10⁵ EL-4 tumour cells injected into the opposing flank (Table 1). In contrast, a challenge with 2×10⁷ EL4 tumour cells was only resisted by mice treated with the B7-H3 and B7-1 combination, indicating that combination treatment generates strong systemic anti-tumour immunity.

Adoptive transfer of 2×10⁸ splenocytes, from mice whose tumours had been injected with B7-H3 or B7-1 plasmids, into recipients bearing established small 0.1 cm diameter EL-4 tumours resulted in rapid and complete tumour regression (P<0.01) (FIG. 7B). However, larger 0.35 cm diameter tumours resisted the affects of splenocytes adoptively transferred from B7-1, B7-H3 treated mice, as well as mice treated with the combination of B7-H3 and B7-1. Nevertheless, tumours in recipients that had received splenocytes from mice treated with a combination of B7-H3 and B7-1 plasmids grew much more slowly (P<0.05) than those which received splenocytes from mice treated with monotherapies (FIG. 7C).

B7-H3 and B7-1 Synergize in Facilitating Initial Tumour Cell Lysis

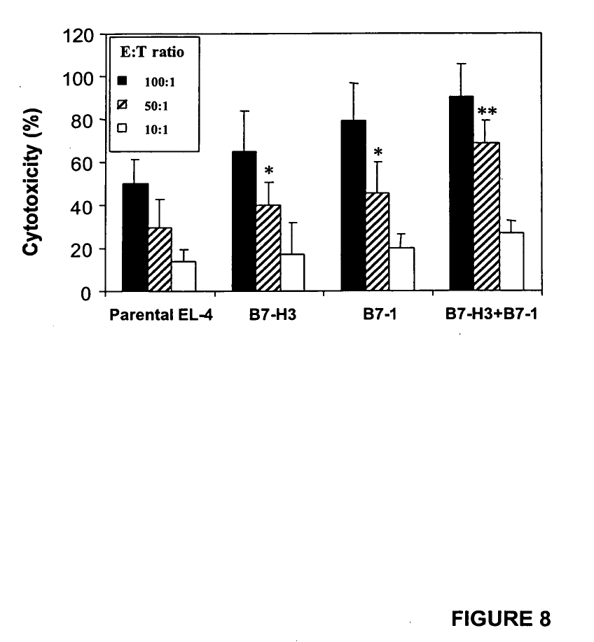

The inventors have previously demonstrated that EL-4 cells transfected with B7-1 are more readily lysed by anti-tumour CTLs than are nontransfected parental EL-4 cells.¹³ To assess whether B7-H3 may also facilitate CTL-mediated tumour cell lysis, either alone or in combination with B7-1, an in vitro CTL killing assay was employed where splenocytes from animals bearing B7-1-treated tumours were mixed with disaggregated EL-4 cells that had been isolated (2 days after gene transfer) from the tumours of animals treated with either B7-H3, B7-1, or a combination of B7-H3 and B7-1. At an effector to target ratio of 50:1, CTL showed significant killing of tumour cells transfected with either B7-1 (P<0.01) or B7-H3 (P<0.05) compared to their ability to kill parental EL-4 cells (FIG. 8). Furthermore, EL-4 cells transfected with a combination of B7-1 and B7H3 were more readily killed than those singly transfected with B7-1 (P<0.01) and B7-H3 (P<0.01).

B7-H3 Immunotherapy Synergizes with a Vascular Attack by Antisense HIF-1α to Eradicate Large Tumours

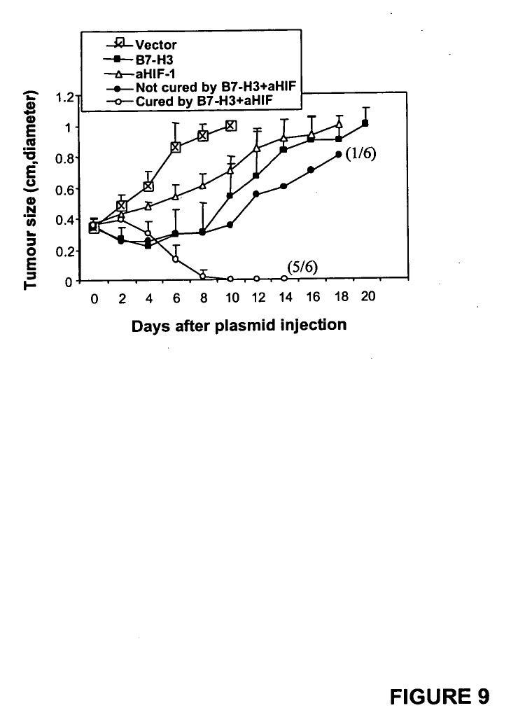

The inventors have previously reported¹⁴ that injection of tumours with plasmids encoding antisense HIF-1α in combination with B7-1-mediated immunotherapy overcomes tumour immune-resistance and leads to the eradication of large tumours. Here the possibility that antisense HIF-1α might synergize with B7-H3 in fighting tumours was considered. Tumours of 0.4 cm in diameter were injected with 100 μg each of the B7-H3, and antisense HIF-1 α plasmids, or with B7-H3 plasmid followed 48 h later by 100 μg antisense HIF-1 α plasmid. As shown in FIG. 9 and Table 1, none of the tumours treated with the monotherapies completely regressed, albeit there was a significant (P<0.01) inhibition of tumour growth. All tumours eventually reached 1 cm in diameter within 2 weeks, and mice had to be euthanased. In contrast, combined gene therapy led to complete regression of tumours in 5 of 6 mice (FIG. 9, Table 1). To determine whether systemic anti-tumour immunity had been generated, mice cured by the combination therapy were rechallenged with 1×10⁶ parental tumour cells. Such mice resisted the challenge, and remained tumour-free (Table 1), indicating that an anti-tumour immune response had developed.

Discussion

This study led to the characterization of a new cDNA clone encoding mouse B7-H3, which encoded a protein identical to a recently reported mouse B7-H3 homologue,¹⁸ except for the substitution of an alanine for a serine in the cytoplasmic domain. This substitution would only be consequential should the B7-H3 cytoplasmic tail, and this site in particular, undergo phosphorylation. In accord with the previous report,¹⁸ transcripts encoding mouse B7-H3 mRNA were found to be widely expressed in a variety of mouse tissues. Transcripts were also expressed in EL-4 tumours, which comprised the tumour model used in the present study, yet were undetectable in in vitro cultured EL-4 cells. Since no anti-B7-H3 antibody is currently available, it is not possible to correlate the latter finding to determine whether B7-H3 is expressed de novo on EL-4 cells in situ, or more likely that it is expressed on cells of the tumour vasculature. In any event, endogenous low level B7-H3 protein expression, if present, appeared to have no significant impact on the growth of EL-4 tumours in situ.

The present study has demonstrated for the first time that mouse B7-H3 can be employed to induce anti-tumour immunity. Gene transfer of mouse B7-H3 was very effective against small EL-4 tumours (<0.3 cm in diameter) causing their complete regression in 50% of cases, but was less effective against larger tumours (>0.3 cm in diameter), whose growth could only be slowed. The regression of large tumours could not be achieved by increasing the dosage of B7-H3 plasmid. Transfection efficiency of large versus small tumours was not the problem, as the Flag-tagged B7-H3 plasmid was espressed at similar levels in large and small tumours. The anti-tumour activity of B7-H3 was comparable with B7-1 which caused the regression of 70% of small tumours. Previous studies have indicated that large tumours are likewise refractory to B7-1 immunogene therapy.¹³⁻¹⁷ The efficacy of B7-1 immunogene therapy appears to be dependent on the inherent antigenicity of the tumour.¹⁹ Whilst the murine EL-4 lymphoma is an immunogenic tumour cell line, EL-4 tumours become increasingly immunosuppressive as they grow larger and begin to express the anti-apoptotic factor survivin, which may render them less susceptible to immune attack. At increased density they upregulate expression of fas ligand,¹³ which has the potential to kill fas-expressing anti-tumour effector T cells. In addition, they secrete TGF-β, which down-regulates anti-tumour immunity by inducing IL-10-mediated development of Th2 responses, and inhibition of Th1 responses.²⁰ Thus, the tumorigenicity of EL-4 lymphomas is suppressed by soluble type II TGF-β therapy.21 B7-H3 immunogene therapy is able to overcome all these various immunosuppressive strategies whilst tumours are a manageable size, and can confer systemic and long-lived anti-tumour immune protection.

B7 family members display temporal and spatial differences in expression, and stimulate both discrete and overlapping costimulatory pathways. The present study surprisingly demonstrates that at the least, timed intratumoral gene transfer of a combination of B7-H3 and B7-1, where one plasmid was injected first followed 48 h later by the other, induces stronger anti-tumour immunity than either B7-H3 or B7-1 monotherapies. Whilst not wishing to be bound by any particular theory, the inventors contemplate that synergy may result from the fact that B7-H3 and B7-1 stimulate different effector cell types, where B7-1 stimulates naive T cells and B7-H3 stimulates effector and memory T cells.

The inventors have also had surprising results in combining B7-H3 therapy with antisense HIF-1α, as is noted from the Examples above. These results are further discussed and the combined B7-H3/antisense HIF-1α therapy covered in a separate patent application filed simultaneously with the present application.

TABLE 1
Assessment of anti-tumour activity and memory response

| | Complete rejection | | Detectable tumour | |
|---|---|---|---|---|
| | of tumours | | after rechallenge | |
| | Tumours | Tumours | 2 × 10⁵ | 2 × 10⁷ |
| Plasmid injected | 0.1-0.25 cm | 0.35-0.4 cm | EL-4 cells | EL-4 cells |
| Empty vector | 0/6 | 0/6 | ND | ND |
| B7-1 | 4/6 | 0/6 | 0/6 | 6/6 |
| B7H3 | 3/6 | 0/6 | 0/6 | 6/6 |
| B7-1 + B7H3 | ND | 3/6 | 0/6 | 0/6 |
| aHIF | 6/6 | 0/6 | 6/6 | ND |
| B7-H3 + aHIF | ND | 5/6 | 0/6 | ND |

Tumours of 0.1-0.25 or 0.35-0.4 cm in diameter established in C57BL/6 mice were injected with the indicated plasmids, and rejection of tumours recorded after two weeks. Mice that were cured of their tumours were rechallenged with either 2 × 10⁵ or 2 × 10⁷ parental tumour cells. Tumour occurrence was recorded after 3 weeks. ND, not done.

The invention has been described herein with reference to certain preferred embodiments, in order to enable the reader to practice the invention without undue experimentation. Those skilled in the art will appreciate that the invention is susceptible to variations and modifications other than those specifically described. It is to be understood that the invention includes all such variations and modifications. Furthermore, titles, headings, or the like are provided to enhance the reader's comprehension of this document, and should not be read as limiting the scope of the present invention.

The entire disclosures of all applications, patents and publications, cited above and below, if any, are hereby incorporated by reference.

The reference to any prior art in this specification is not, and should not be taken as, an acknowledgment or any form of suggestion that that prior art forms part of the common general knowledge in the field of endeavour to which the invention relates.

Unless the context clearly requires otherwise, throughout the description and the claims, the words “comprise”, “comprising” and the like, are to be construed in an inclusive sense as opposed to an exclusive or exhaustive sense, that is to say, in the sense of “including, but not limited to”.

Bibliographic Details

1. Chambers C A, Allison J P. Costimulatory regulation of T cell function. Curr Opin Cell Biol 1999; 11: 203-210.
2. Liang L, Sha W C. The right place at the right time: novel B7 family members regulate effector T cell responses. Curr Opin Immunol 2002; 14: 384-390.
3. Coyle A, Gutierrez-Ramos J. The expanding B7 superfamily: Increasing complexity in costimulatory signals regulating T cell function. Nat Immunol 2001; 2: 203-209.
4. Henry J, Miller M, Pontarotti P. Structure and evolution of the extended B7 family. Immunol Today 1999; 20: 285-288.
5. Yoshinaga S K et al. T cell co-stimulation through B7RP-1 and ICOS. Nature 1999; 402: 827-831.
6. Hutloff A et al. ICOS is an inducible T-cell co-stimulator structurally and functionally related to CD28. Nature 1999; 397: 263-266.
7. Freeman G et al. Engagement of the PD-1 immunoinhibitory receptor by a novel B7. family member leads to negative regulation of lymphocyte activation. J Exp Med 2000; 192: 1027-1034.
8. Latchman Y et al. PD-L2 is a second ligand for PD-1 and inhibits T cell activation. Nat Immunol 2001; 2: 261-268.
9. Tseng S Y et al. B7-DC a new dendritic cell molecule with potent costimulatory properties for T cells. J Exp Med 2001; 193: 839-846.
10. Nishimura H et al. Development of lupus-ike autoimmune diseases by disruption of the PD-1 gene encoding an ITIM motif-carrying immunoreceptor. Immunity 1999; 11: 141-151.
11. Nishimura H et al. Autoimmune dilated cardiomyopathy in PD-1 receptor-deficient mice. Science 2001; 291: 319-322.
12. Chapoval A et al. B7-H3: A costimulatory molecule for T cell activation and IFN-gamma production. Nat Immunol 2001; 2: 269-274.
13. Kanwar J, Berg R, Lehnert K, Krissansen G W. Taking lessons from dendritic cells: Multiple xenogeneic ligands for leukocyte integrins have the potential to stimulate anti-tumour immunity. Gene Ther 1999; 6: 1835-1844.
14. Sun X et al. Gene transfer of antisense hypoxia inducible factor-1α enhances the therapeutic efficacy of cancer immunotherapy. Gene Ther 2001; 8: 638-645.
15. Sun X et al. Angiostatin enhances B7.1-mediated cancer immunotherapy independently of effects on vascular endothelial growth factor expression. Cancer Gene Ther 2001; 8: 719-727.
16. Kanwar J R, Shen W P, Berg R, Krissansen G. W. Effect of survivin antagonists on the growth of established tumours and B7.1 immunogene therapy. J Natl Cancer Inst 2001; 93:1541-1552.
17. Kanwar J R et al. Vascular attack by 5,6-dimethylxanthenone-4-acetic acid combined with B7.1-mediated immunotherapy overcomes immune-resistance and leads to the eradication of large tumours. Cancer Res 2000; 61: 1948-1956.
18. Sun M et al. Characterization of mouse and human B7-H3 genes. J Immunol 2002; 168: 6294-6297.
19. Chen L et al. Tumour immunogenicity determines the effect of B7 costimulation on T cell-mediated tumour immunity. J Exp Med 1994; 179: 523-532.
20. Maeda H, Shiraishi A. TGF-beta contributes to the shift toward Th2-type responses through direct and IL-10-mediated pathways in tumour-bearing mice. J Immunol 1996; 156: 73-78.
21. Won J et al. Tumorigenicity of mouse thymoma is suppressed by soluble Type II transforming growth factor β receptor therapy. Cancer Res 1999; 59: 1273-1277.
22. Tannenbaum C S, Hamilton T A. Immune-inflammatory mechanisms in IFNgamma-mediated anti-tumour activity. Sem Cancer Biol 2000; 10: 113-123.
23. Wilson J L et al. NK cell triggering by the human costimulatory molecules CD80 and CD86. J Immunol 1999; 163: 4207-4212.
24. PCT/IUS01/41430
25. PCT/NZ00/00098
26. Yang J-P, Huang I. Direct gene transfer to mouse melanoma by intratumor injection of free DNA. Gene Therapy 1996; 3: 542-548.
27. Antonia S J. Seigne J. Diaz J. Muro-Cacho C. Extermann M. Farmelo M J. Friberg M. Alsarraj M. Mahany J J. Pow-Sang J. Cantor A. Janssen W. Phase I trial of a B7-1 (CD80) gene modified autologous tumor cell vaccine in combination with systemic interleukin-2 in patients with metastatic renal cell carcinoma. [Clinical Trial. Clinical Trial, Phase I. Evaluation Studies. Journal of Urology. 167:1995-2000, 2002.
28. Cavallo F. Martin-Fontecha A. Bellone M. Heltai S. Gatti E. Tomaghi P. Freschi M. Forni G. Dellabona P. Casorati G. Co-expression of B7-1 and ICAM-1 on tumors is required for rejection and the establishment of a memory response. [Journal Article] European Journal of Immunology. 25(5):1154-62, 1995.
29. Rafiee, M., Kanwar, J. R., Berg, R. W., Lehnert, K., Lisowska, K., and Krissansen, G. W. Induction of systemic antitumour immunity by gene transfer of mammalian heat shock protein 70.1 into tumours in situ. Cancer Gene Ther. 8: 974-981, 2001.
30. Koumarianou A A, Hudson M, Williams R, Epenetos A A, Stamp G W H. Development of a novel bi-specific monoclonal antibody approach for tumour targeting. Brit J Cancer 1999; 81:431-439.
31. McHugh R S. Ahmed S N. Wang Y C. Sell K W. Selvaraj P. Construction, purification, and functional incorporation on tumor cells of glycolipid-anchored human B7-1 (CD80). [Journal Article] Proceedings of the National Academy of Sciences of the United States of America. 92(17):8059-63, 1995.
32. Greenfield E A. Nguyen K A. Kuchroo V K. CD28/B7 costimulation: a review. Critical Reviews in Immunology. 18(5):389-418, 1998
33. Freeman, G. J., Freedman, A. S., Segil, J. M., Lee, G., Whitman, J. F. and Nadler, L. M. B7, a new member of the Ig superfamily with unique expression on activated and neoplastic B cells. J. Immunol. 143 (8), 2714-2722 (1989).
34. Burgstaller P. Jenne A. Blind M. Aptamers and aptazymes: accelerating small molecule drug discovery. Current Opinion in Drug Discovery & Development. 5(5):690-700, 2002.
35. Lehnert, K., Print, C. G., Yang, Y., and Krissansen, G. W. Mucosal addressin cell adhesion molecule-1 (MAdCAM-1) costimulates T cell proliferation exclusively through integrin α4β7 (LPAM-1), whereas VCAM-1 and CS-1 peptide use α4β1 (VLA-4): Evidence for “remote” costimulation and induction of hyperresponsiveness to B7 molecules. (1998) Eur. J. Immunol. 28: 3605-3615.
36. PROTEINS—STRUCTURE AND MOLECULAR PROPERTIES, 2nd Ed., T. E. Creighton, W. H. Freeman and Company, New York (1993)
37. POSTTRANSLATIONAL COVALENT MODIFICATION OF PROTEINS, B. C. Johnson, Ed., Academic Press, New York, pgs. 1-12 (1983)
38. Seifter et al., Meth Enzymol 182:626-646 (1990)
39. Rattan et al., Ann NY Acad Sci 663:48-62 (1992)
40. Ponnazhagan S, Hoover F. Delivery of DNA to tumor cells in vivo using adeno-associated virus. Methods Mol Biol. 2004;246:237-43
41. Ponnazhagan S, Curiel D T, Shaw D R, Alvarez R D, Siegal G P. Adenoassociated virus for cancer gene therapy. Cancer Res 2001;61:6313-6321
42. Xu, R., Sun, X., Tse, L. Y., Li, H., Chan, P. C., Xu, S., Xiao, W., Kung, H. F., Krissansen, G. W. Fan S T. Long-term expression of angiostatin suppresses metastatic liver cancer in mice. Hepatology 37:1451-60, 2003.
43. Keith L, Knutson K L, Almand B, Mankoff D A, Schiffmnan K, Disis M L. Adoptive T-cell therapy for the treatment of solid tumours. Expert Opin. Biol. Ther. (2002) 2(1): 55-66.
44. Singh N P, Yolcu E S, Taylor D D, Gercel-Taylor C, Metzinger D S, Dreisbach S K, Shirwan H. A novel approach to cancer immunotherapy: tumor cells decorated with CD80 generate effective antitumor immunity. Cancer Res. 2003 63:4067-73
45. Antonia S J, Seigne J D. World J Urol. 2000; 18:157-63. B7-1 gene-modified autologous tumor-cell vaccines for renal-cell carcinoma.

1. A method of treating tumors in a subject, the method comprising at least the steps of administering:
an effective amount of an agent adapted in use to increase B7-H3; and
an effective amount of an agent adapted in use to increase B7-1.

2. A method as claimed in claim 1 wherein the agent adapted to increase B7-H3 is chosen from the group consisting: B7-H3 or a functional equivalent thereof; and, a nucleic acid vector adapted to express in use B7-H3 or a functional equivalent thereof.

3. A method as claimed in claim 2 wherein the functional equivalent of B7-H3 is a polypeptide chosen from the group consisting: a polypeptide having at least approximately 40% amino acid sequence identity to B7-H3 of GenBank accession number AF302102; or a polypeptide which is a fragment of the B7-H3 of GenBank accession number AF302102; and, wherein, the functional equivalent retains the ability to bind to natural ligands of B7-H3 involved in enhancing immune responses mediated by T cells.

4. A method as claimed in claim 1 wherein the agent adapted to increase B7-1 is chosen from the group consisting: B7-1 or a functional equivalent thereof; and, a nucleic acid vector adapted to express in use B7-1 or a functional equivalent thereof.

5. A method as claimed in claim 4 wherein the functional equivalent of B7-1 is a polypeptide chosen from the group consisting: a polypeptide having at least approximately 40% amino acid sequence identity to the B7-1 of GenBank accession number NM_{—}005191 or P33681; or a polypeptide which is a fragment of the B7-1 of GenBank accession number NM_{—}005191 or P33681; and, wherein, the functional equivalent retains the ability to bind to natural ligands of B7-1 involved in enhancing immune responses mediated by T cells.

6. A method as claimed in claim 1 wherein the agents are administered intratumorally.

7. A method as claimed in claim 1 wherein the agents are administered systemically.

8. A method as claimed in claim 1 wherein the agents are administered sequentially in any order.

9. A method as claimed in claim 1 wherein the agents are administered simultaneously.

10. A method as claimed in claim 1 wherein the subject is a mammal.

11. A method as claimed in claim 10 wherein the mammal is a human.

12. A method of inducing anti-tumour immunity comprising at least the step of administering to a subject an effective amount of an agent adapted in use to increase B7-H3.

13. A method as claimed in claim 12 wherein said agent is chosen from the group consisting: B7-H3 or a functional equivalent thereof; and, a nucleic acid vector adapted to express in use B7-H3 or a functional equivalent thereof.

14. A method of treating tumors in a subject, the method comprising at least the steps of:
conducting a method as claimed in claim 1 or:
isolating one or more immune cells from the subject;
expanding the one or more immune cells in vitro;
returning said immune cells to the subject.

15. A method as claimed in claim 14 wherein the one or more immune cells are splenocytes, lymph node lymphocytes, or tumour-infiltrating lympocytes.

16. A method as claimed in claim 14 wherein the immune cells are returned to the subject by injection.

17. A method of treating tumours comprising at least the steps of:
isolating one or more tumour cells from a tumour-bearing subject;
exposing one or more tumour cells to an effective amount of an agent adapted in use to increase B7-H3 and an agent adapted in use to increase B7-1; and
returning the one or more cells to the subject.

18. A method as claimed in claim 17 wherein the one or more tumour cells are exposed to said agents simultaneously.

19. A method as claimed in claim 17 wherein the one or more tumour cells are exposed to said agents sequentially.

20. A method as claimed in claim 17 wherein the agent adapted to increase B7-H3 is chosen from the group consisting: B7-H3 or a functional equivalent thereof; a nucleic acid expression vector adapted to express in use B7-H3 or a functional equivalent thereof.

21. A method as claimed in claim 17 wherein the agent adapted to increase B7-1 is chosen from the group consisting: B7-1 or a functional equivalent thereof; and, a nucleic acid vector adapted to express in use B7-1 or a functional equivalent thereof.

22. A method as claimed in claim 20 wherein the one or more isolated tumour cells are exposed to said agents by transfection with a nucleic acid vector adapted in use to express B7-H3 and B7-1, or functional equivalents thereof, or separate nucleic acid vectors adapted in use to express each separately.

23. A method as claimed in claim 17 wherein the one or more tumour cells are returned to the subject via injection.

24. A composition comprising at least an agent adapted in use to increase B7-H3 and an agent adapted in use to increase B7-1 together with one or more pharmaceutically acceptable carriers, diluents or excipients.

25. A composition as claimed in claim 24 wherein the agent adapted to increase B7-H3 is chosen from the group consisting: B7-H3 or a functional equivalent thereof; and a nucleic acid vector adapted to express B7-H3 or a functional equivalent thereof.

26. A composition as claimed in claim 24 wherein the agent adapted to increase B7-1 is chosen from the group consisting: B7-1 or a functional equivalent thereof; and a nucleic acid vector adapted to express B7-1 or a functional equivalent thereof.

27. A kit comprising at least:
an agent adapted in use to increase B7-H3; and separately,
an agent adapted in use to increase B7-1.

28. A murine B7-H3 nucleic acid chosen from the group consisting:
The nucleic acid represented by SEQ ID NO: 1; and
A nucleic acid encoding the murine B7-H3 of SEQ ID NO:2.

29. A murine B7-H3 chosen from the group consisting:
The polypeptide represented by SEQ ID NO:2; and,
The polypeptide represented by SEQ ID NO:2 without the signal peptide.

30. A method of treating tumors in a subject, the method comprising at least the steps of:
conducting a method as claimed in claim 12:
isolating one or more immune cells from the subject;
expanding the one or more immune cells in vitro;
returning said immune cells to the subject.

31. A method as claimed in claim 30 wherein the one or more immune cells are splenocytes, lymph node lymphocytes, or tumour-infiltrating lympocytes.

32. A method as claimed in claim 30 wherein the immune cells are returned to the subject by injection.

33. A method as claimed in claim 30 wherein the one or more isolated tumour cells are exposed to said agents by transfection with a nucleic acid vector adapted in use to express B7-H3 and B7-1, or functional equivalents thereof, or separate nucleic acid vectors adapted in use to express each separately.
